(19) United States
(12) Patent Application Publication
(10) Pub. No.: US 2010/0266523 A1
(43) Pub. Date: Oct. 21, 2010

(54) COMPOSITIONS CONTAINING FLAVANOID POLYPHENOL DERIVATIVES, AND APPLICATIONS THEREOF IN CONTROLLING DISEASES AND AGEING OF LIVING ORGANISMS

(76) Inventor: Joseph Vercauteren, Castelnau-le-Lez (FR)

Correspondence Address: NIXON & VANDERHYE, PC, 901 NORTH GLEBE ROAD, 11TH FLOOR, ARLINGTON, VA, 22203, US

(21) Appl. No.: 12/734,678

(22) PCT Filed: Nov. 17, 2008
(86) PCT No.: PCT/IB2008/054814

§ 371 Date: May 14, 2010

(30) Foreign Application Priority Data
Nov. 15, 2007 (FR) 07/08021

(51) Int. Cl. A61K 31/353; C07C 39/15; C07C 39/21; C08G 65/38; C07D 311/04; A61K 31/775; A61K 8/49; A61Q 19/00

(52) U.S. Cl. 424/78.03; 568/729; 528/205; 528/219; 549/400; 528/125; 514/456; 424/78.38

(57) ABSTRACT

The invention relates to compositions of polyphenol derivatives, characterised in that said polyphenols contain monomers, oligomers or polymers of units of the formula (I), wherein said units are characterised by the simultaneous presence of a phloroglucinol-type core (core A) and of a catechol-type core (core B) bonded together by a segment of 3 carbons such as C, said derivatives being over-activated in terms of nucleophilic power by the alkylation of at least one phenol function of each constituent monomer unit, and stabilised by the esterification of all the others with mixtures of fatty acids in proportions representing those of vegetable oils mainly consisting of AGI. These compositions can particularly be used in cosmetics, nutrition and therapy.

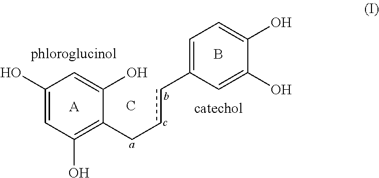

The invention relates to compositions of flavonoid polyphenol derivatives for preventing and controlling the majority of pathologies and the aging of living organisms and tissues. It also relates to a process for preparing these compositions, and to their applications, especially in the fields of cosmetology, dietetics, and therapeutics.

More than fifty years ago, a theory developed whereby the aging of the human body is a result of the accumulation of multiple damage caused to the tissues by free-radical species or oxidizing chemical reactivities.

In the middle of the 1950s, after numerous studies on rubber, the chemist Harman observed that preventing the formation of free radicals was the most certain way to prevent its degradation and cracking. By analogy, he then suggested that the aging of human tissues (appearance of wrinkles in the skin, for example) might be caused by the “abnormal” formation, within cells, of highly reactive chemical species, and especially free radicals, and by the reaction sequences that they triggered.

Reactive oxygen species (ROS) are formed at the mitochondrial level by uncontrolled “transfer” of one or more electrons to oxygen (ROS: superoxide anion, peroxides, peroxynitrites, free radicals, etc.).

These ROS subsequently propagate to the other cellular compartments or to the cytoplasm, depending on their water/fat solubility, where they produce considerable damage.

In this kind of context, the search for active substances for controlling aging has been conducted, over the recent decades, on the basis of their capacity to break the chain oxidation reactions, in other words to prevent the oxidative stress. In effect, any substance capable of interacting with the ROS will lessen the deleterious effects and, over the longer term, will have a positive impact on health, and, for the same reasons, will slow down aging and the development of the main pathologies. Such substances are free radical scavengers (capable of delivering a single electron at a time) and/or antioxidants (transfer of two electrons at the same time) such as vitamins (E and C) and polyphenols.

However, the damage caused by the aging of the body or accompanying the major pathologies is unlikely to be solely the consequence of poor control of the flow of electrons owing to “leaks” of the mitochondrial metabolism and of intracellular ROS, but is also likely to involve other sources of potential deleterious effects, involving the Maillard reaction and carbonyl stress.

In carbonyl stress, the carbonyl (aldehyde) function of glucose exerts its electrophilic properties with regard to the nucleophilic residues of proteins (amines, thiols, etc.): this is the starting point for carbonyl stress, which is amplified by formation of propagators.

The chemical species produced, or glycation products, are considered to be end products: these are AGEs, for Advanced Glycated End-Products, in which glucose or its fragments are joined irreversibly to the amino acid residues.

The Maillard reactions which take place increase, at the same time, the reducing capacity of the sugars and of their derivatives. The dicarbonyl compounds which form acquire an oxidizability which is much greater even then their precursors, and readily transfer their electrons to oxygen, for example. Starting from the superoxide anion formed initially, the same sequence of ROS as in the case of intracellular stress is produced. Accordingly, the carbonyl stress is coupled with a second type of an oxidative stress.

In contradistinction to the mechanisms set out above for the ROS of mitochondrial origin, this new oxidative stress occurs outside the cells, within the extracellular matrix. It therefore affects the amino acids or protein residues of this matrix, and especially the fibers of collagen and of elastin. This oxidative stress, which is particularly significant in view of the fact that the enzymatic protection systems are not as effective as those situated within the cell, results in an increase in the alkylation phenomena which add to the glycation and glycoxidation products resulting from carbonyl stress.

Accordingly, carbonyl stress, coupled with an extracellular oxidative stress, is at least as significant as the intracellular oxidative stress in the development of aging and the establishment of the tissue alternations that accompany the major pathologies.

The study by the inventors of the phenomena leading to tissue aging has therefore led them to a more extended appreciation of the biochemical mechanisms responsible for such aging and to develop new concepts permitting the definition of new biological targets of complementary action for their more effective control.

Their research has therefore resulted in modification to the structure of polyphenols having antioxidant and free-radical-scavenger properties, such as those which make up plant extracts, in order to provide them with greater abilities to likewise scavenge carbonyl stressors.

It is therefore an object of the invention to provide new compositions of polyphenol derivatives that constitute overactivated polyphenols which both are capable of acting very efficiently on a larger number of biological targets and are stabilized.

Another object of the invention is to provide a process for obtaining such polyphenol derivatives from plant extract polyphenols.

In accordance with yet a further aspect, the invention aims to exploit properties of these polyphenol compositions of flavonoid type in cosmetology, dietetics, and therapeutics.

The polyphenol derivative compositions of the invention are characterized in that said polyphenols comprise monomers, oligomers or polymers of units conforming to the formula (I):

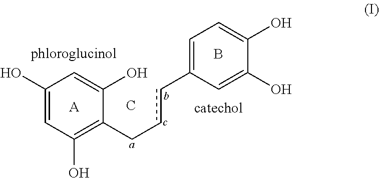

These units are characterized by the simultaneous presence of a phloroglucinol nucleus (nucleus A) and of a catechol nucleus (nucleus B), which are joined to one another by a 3 carbon linkage such as C.

In the most common case, in these units, the A ring is fused to a further oxygen-containing heterocycle by formation of a bond of one of its oxygens with the carbon b of the segment C (case of the flavonoid backbone) of formula (II)

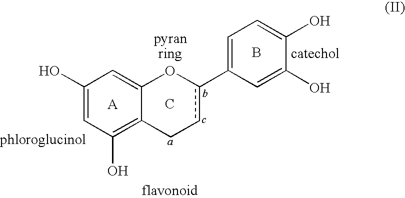

The 3 carbons of the segment C may be sp2 hybridized (double bond between b and c and carbonyl at a) as is the case for quercetin of formula (III)

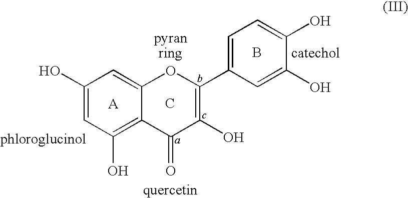

or contain a double bond between a and c and carbonyl at b, as in cyanidol of formula (IV)

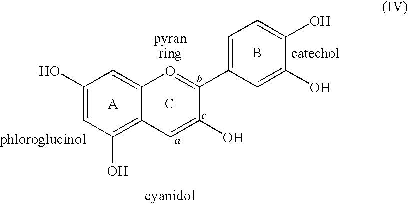

or its carbon a may be sp3 hybridized alone, or, lastly, may be all 3 sp3 hybridized, as in the case of catechin of formula (V)

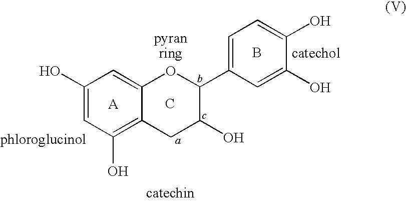

The carbon a of the segment C then serves usually as a point of attachment with the A rings of the other units to form the oligomers or the polymers.

Said derivatives are overactivated, with regard to their nucleophilic power, by alkylation of at least one phenolic function of each unit, and are stabilized by esterification, with mixtures of predominantly unsaturated fatty acids (UFA), of all of the others which have remained free.

Generally speaking, the specific substitutions of the derivatives in the compositions of the invention lead to a modulation of their activity and enable them, at the same time and specifically, to inhibit the principal mechanisms involved in the primary pathologies and the aging as set out above.

Advantageously, the number of —O-alkyl groups per molecule is not equal to the number of hydroxyls present on average per unit, and preferably is 1 or 2, more especially equal to 1.

The alkyl group or groups are more particularly methyl, isopropyl or tert-butyl groups.

Effective stabilization is obtained by formation of FA esters between the hydroxyl (alcoholic and phenolic) functions that have remained free after alkylation (of 2 to 3, preferably 3), and fatty acids from vegetable oils characterized by their particularly high levels of unsaturated fatty acids (UFA). The oils are selected for their favorable effect on health. Advantageously, the active substances obtained then contain proportions of unsaturated fatty acids that are identical with those of the oils from which they originate.

Said esters preferably comprise the mixtures of acyl radicals R from the fatty acids of olive oil (Olea europea) or grapeseed oil (Vitis vinifera).

The radicals in question are more especially radicals R of saturated fatty acids (SFA=stearic acid; 7-8%), of monounsaturated fatty acids (MUFA=oleic acid; 55-75%), and of essential polyunsaturated fatty acids (PUFA; 15-18%): diunsaturated (linoleic acids) and triunsaturated (linolenic acids) of the ω-6 and ω-3 series, which are present in the derivatives of the invention in proportions identical to those of the oils which produce a maximum benefit for health, according to the data obtained from epidemiology.

This stabilization makes it possible, furthermore, to protect the overactivated flavonoid polyphenols from certain premature destruction (oxidation in the air or in the light), while giving them a lipophilic character in order to enhance their chances of being absorbed and of acting.

Advantageously, however, this stabilization is temporary, and is no longer effective when the derivatives are put in place to act, so as to restore to them all of their antioxidant power. The stabilization must therefore be reversible by the simple action of the biological systems to which the stabilizing groups are then exposed, and especially enzymes such as lipases, esterases or proteases.

More specifically, the invention relates to compositions characterized in that said unitary derivatives conform to the formula (VI):

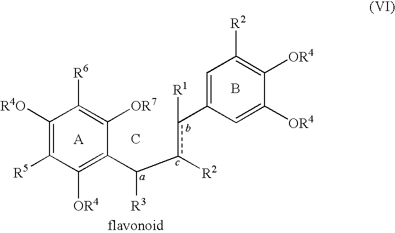

in which
R¹ is a hydrogen or the junction point at R⁷ of a single unit,
R² is a hydrogen, or an O-acyl radical of a fatty acid from a vegetable oil, represented by R as defined above.
R³ is a hydrogen, a carbonyl or the junction point at R⁵ or at R⁶ of another unit,
R⁴ is an alkyl radical, or an acyl radical of a fatty acid of a vegetable oil, represented by R as defined above,
R⁵ is a hydrogen or the junction point at R³ of another unit, directly or through a carbon entity (methylene, methylmethine, etc.),
R⁶ is a hydrogen or the junction point at R³ of another unit, directly or through a carbon entity (methylene, methylmethine, etc.),
R⁷ is an alkyl radical or an acyl radical of a fatty acid of a vegetable oil, represented by R as defined above, or the junction point at R¹ of the same unit,
   and the diastereoisomers and regioisomers of these moieties.

As an example, it is possible to give the derivatives of the dimer of catechin (B3) and of the trimer of epicatechin (C2), of formulae (VII) and (VIII):

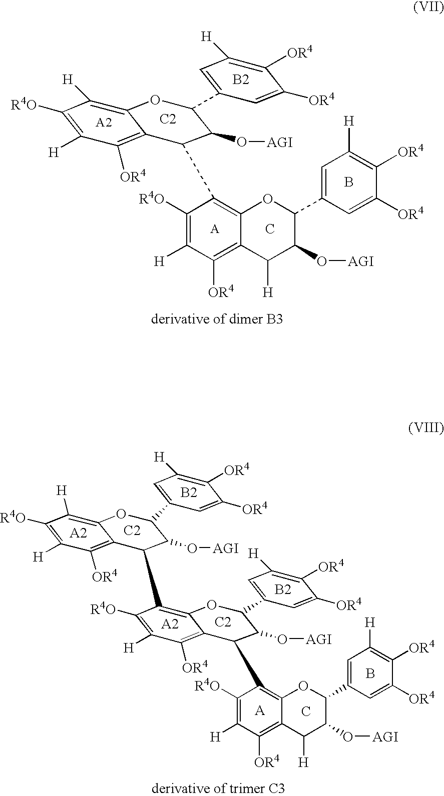

According to one preferred embodiment of the invention, the derivatives defined above correspond to plant extract derivatives which have been alkylated and then stabilized. They therefore have the structures of the polyphenols present as a mixture in these plant extracts.

More particularly these are plant extracts of vine, of fermented or green tea, of fresh or roasted cocoa beans, or of pine.

The vine extracts are obtained from grape seeds or grape marcs.

In accordance with the invention, the polyphenol derivative compositions defined above are obtained by reacting the corresponding polyphenol compositions
in a first step, with an alkylating agent under conditions allowing substitution of an alkyl group for the hydrogen of at least 1 phenolic OH group per constituent monomeric unit of each molecule, preferably of 1 to 2, and
in a second step, with an acylating agent, especially an acid anhydride or acid chloride, under conditions allowing substitution by a mixture of acyl radicals —COR liberated by the acylating agent, R being as defined above, for the hydrogen of the —OH groups which are still free after alkylation.

The alkylation reaction employs reactants which are available commercially, such as halides (iodides, bromides, etc.) or sulfuric esters, in a proportion of one-and-a-half chemical equivalents. They are added slowly to a solution of the polyphenol extract in an aprotic solvent (anhydrous acetone, for example), and in the presence of an inorganic base (potassium carbonate, etc.), which is heated at reflux, with stirring and under an inert atmosphere (nitrogen, argon, ideally).

The alkylation reaction is halted, after cooling, by addition of a dilute acid (hydrochloric acid, for example) until an acid pH is obtained. Stirring is continued for 45 additional minutes, approximately. The reaction mixture is concentrated under vacuum (evaporation of the solvent). The aqueous phase is extracted with an equal volume of immiscible solvent (such as ethyl acetate, dichloromethane, etc.), which is itself washed with two equivalent volumes of distilled water (until neutrality). This organic phase is dried over anhydrous sodium sulfate and then filtered and evaporated under reduced pressure to leave the residue of the alkylated polyphenols.

The acylating agent is prepared from a vegetable oil by a process comprising:
the saponification of the glycerides of the vegetable oil, followed by an acidification,
activation by dehydration where the acylating agent is an acid anhydride, or by chloridation where it is an acid chloride, although other derivatives imparting the same activating effect may be used (transesterification, enzymatic acylation, as appropriate).

The saponification reaction is performed in aqueous phase in the presence of an alkaline agent such as potassium hydroxide in an at least stoichiometric amount, preferably at the reflux temperature. The solution is then brought to acid pH by addition of inorganic acid, then extracted with an organic solvent so as to isolate the mixture of the free acids formed during the reaction.

The dehydration reaction takes place at reflux, in the presence of a solvent capable of producing an azeotrope with water, so as to allow it to be removed in line with its formation. Toluene, for example, is used, and the water is trapped by a Dean Stark system.

The chloridation reaction is conducted in the presence of a solvent capable of dissolving the free fatty acids. It is catalyzed by Lewis base and carried out by slow addition of the chloridating agent, at a controlled temperature, close to 0° C. When the addition is ended, stirring is continued at the ambient temperature and the reaction mixture is then concentrated by evaporation under vacuum, and the chlorides are purified by distillation.

Advantageously:
the solvent used for the chloridation is dichloromethane or chloroform, for example, provided it is not stabilized by an alcohol,
the chloridating agent is, for example, thionyl chloride or oxalyl chloride,
the catalyst may be dimethylformamide,
the acyl chlorides are purified by distillation under a high vacuum, in a “ball oven” (Kugelrohr).

The acylation reaction is usually carried out in the presence of a solvent which allows solubilization, even partial solubilization, of the alkylated polyphenol compounds resulting from the alkylation reaction described above.

Appropriate solvents are selected from halogen derivatives such as dichloromethane, chloroform or 1,2-dichloroethane, or nitrogen derivatives such as pyridine, or even hexane, depending on the alkylated compounds to be dissolved.

The alkylated polyphenol derivatives, in solution in the selected reaction solvent, and advantageously admixed with a basic catalysis agent (for example, triethylamine or pyridine), are placed under stirring and in an inert atmosphere (argon, nitrogen).

Four equivalents of FA anhydrides or chlorides, as prepared above, are used as acylating agents. They are added dropwise, in solution in the solvent for the reaction, unless that solvent is pyridine alone. Where pyridine is both the solvent and the basic catalyst, an “inverse” addition is operated. This involves the solution of the polyphenol derivatives being added dropwise to the acylpyridinium compounds formed beforehand.

One alternative which may be employed involves adding, with vigorous stirring, a basic aqueous phase (Na₃PO₄, K₃PO₄) to the organic solution (CHCl₃, CH₂Cl₂) of the alkylated polyphenol derivatives and of the acylating agents, thus producing Schotten-Baumann conditions.

Whatever procedure is adopted, the reaction is carried out preferably at ambient temperature, for a time of approximately 7 to 8 hours.

The esterified derivatives thus formed are purified by addition of acidulated water (HCl, qs acid pH), then by a number of washes of the organic phase with distilled water. After drying over sodium sulfate, the solution is filtered and then evaporated to dryness to yield the stabilized and alkylated active flavonoid substances.

The dual-effect active substances of the invention, capable of trapping not only the ROS, irrespective of their intracellular or extracellular origin, but also the dicarbonyl compounds (antiglycation and anti-AGEs), are of great interest as the most comprehensive and most effective means to date for combating skin aging.

The compositions of the invention are therefore particularly appropriate for the production of cosmetic preparations.

In these preparations, the compositions are combined with vehicles which are appropriate for external use. Advantageously, their fat-soluble nature favors their incorporation into the product forms that are commonly used in cosmetology.

The invention is therefore directed to cosmetic compositions characterized in that they comprise an amount effective for controlling skin aging of one or more compositions of flavonoid polyphenol derivatives as defined above in combination with inert vehicles which are appropriate for external use.

These compositions take a form appropriate for topical administration, such as cream, ointment, emulsion, gel, liposomes, lotion.

They contain from 0.5% to 5% of active product, preferably from 2% to 3%.

The invention also relates to a method of preventing skin aging, characterized by the application to the skin, or the ingestion, of one or more cosmetic compositions as defined above.

According to another aspect of great interest, the compositions of the invention can be used in dietetics. By virtue especially of their anti-free-radical and carbonyl-compound-scavenging properties, they ensure better preservation of foods. Moreover, they generally constitute a provider of vitamin factor. They are therefore added with advantage to drinks, as for example to fruit juices, tonic drinks, to dairy products and derivatives such as butter.

They can also be used as they are in liquid form, or else as granules or the like, gels or in paste form, incorporated, for example, into confectionery such as fruit gums, candies, chewing gums.

The properties of the compositions of the invention are also advantageously exploited for use as medicaments.

The invention thus also relates to pharmaceutical compositions characterized in that they comprise a therapeutically effective amount of at least one composition as defined above, in combination with a pharmaceutically acceptable vehicle.

These compositions advantageously take a form appropriate for—in particular—oral, topical or parenteral administration.

Accordingly, for oral administration, the compositions take the form more particularly of solutions, tablets, gel capsules or syrups.

For topical administration, the compositions take the form of cream, ointments, gels, lotions or patches.

For parenteral administration, the compositions take the form of a sterile or sterilizable injectable solution.

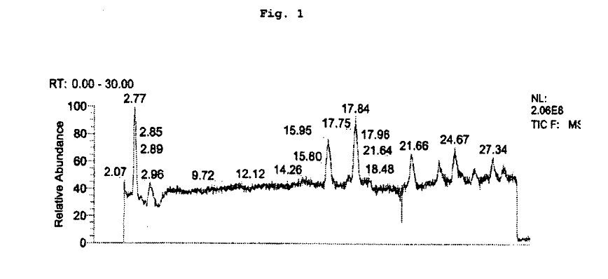

Other characteristics and advantages of the invention are given, by way of illustration, in the examples which follow, in which reference is made to FIGS. 1 to 11, which represent respectively:

FIG. 1: the HPLC-ESI-MS (TIC) chromatogram of O-methylated catechins,

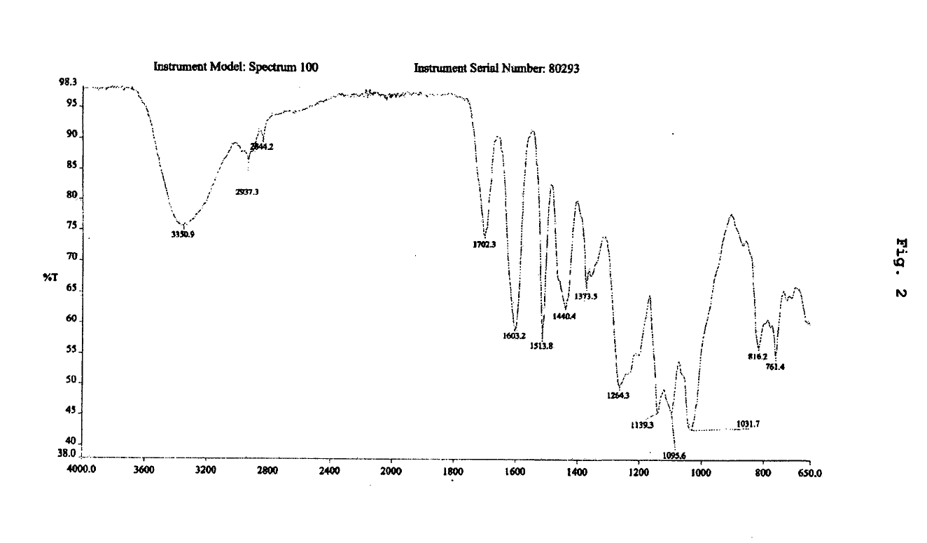

FIG. 2: the FT-IR spectrum, in ATR mode, of alkylated (methylated) grape seed flavanolic polyphenols,

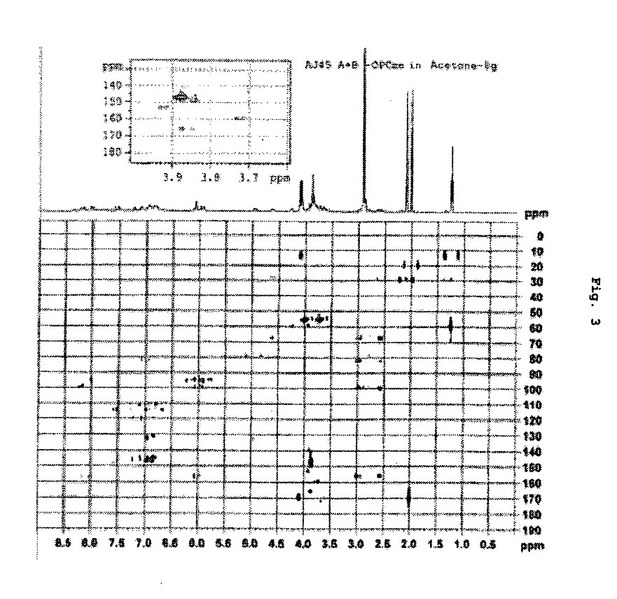

FIG. 3: the ¹H-¹³C HMBC 2D NMR spectrum (500 MHz) of grape seed flavanolic polyphenols alkylated with dimethyl sulfate,

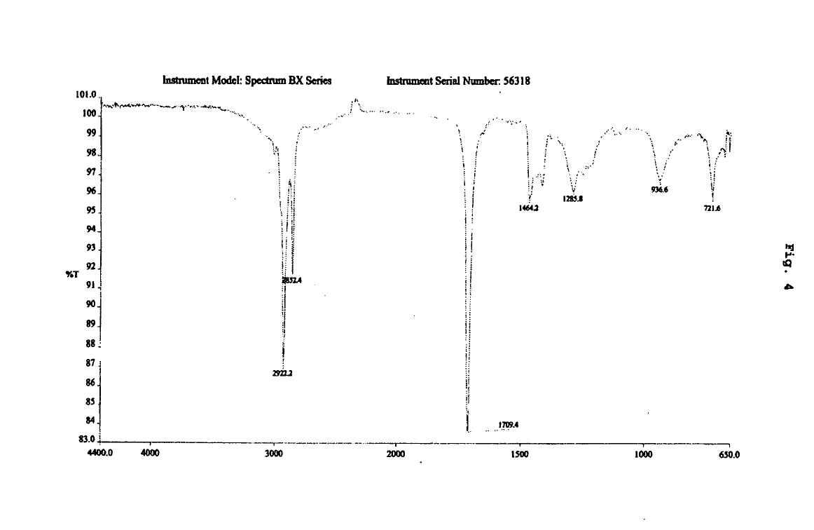

FIG. 4: the FT-IR spectrum of the fatty acids obtained from the saponification of a “virgin” olive oil, in ATR mode,

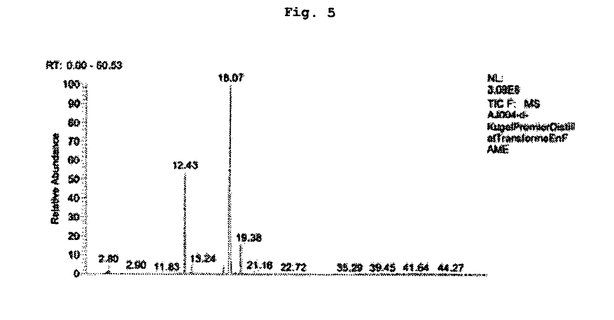

FIG. 5: the gas chromatogram, detected by mass spectrometry (GC-DSQ2), of the methyl esters prepared from olive FA chlorides,

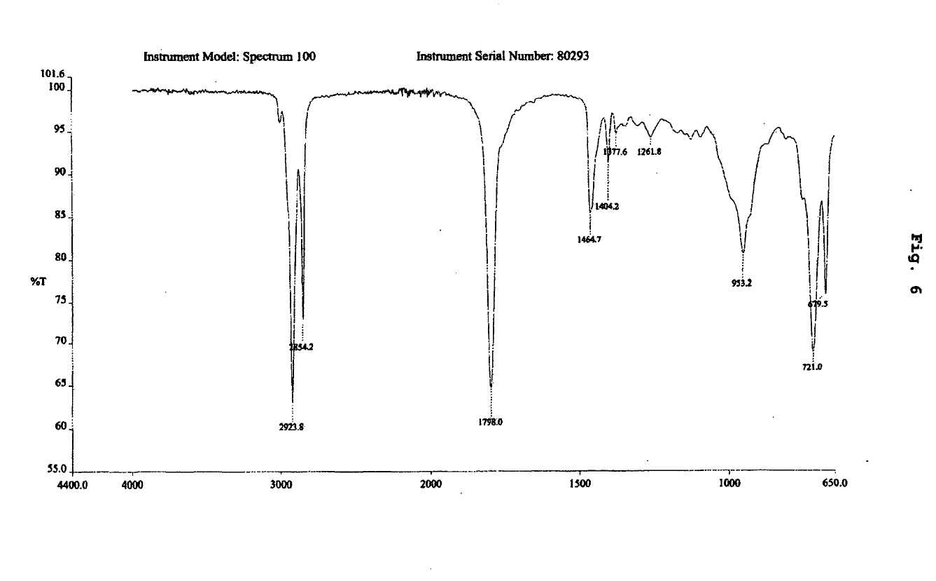

FIG. 6: the FT-IR spectrum of olive FA chlorides (in ATR mode),

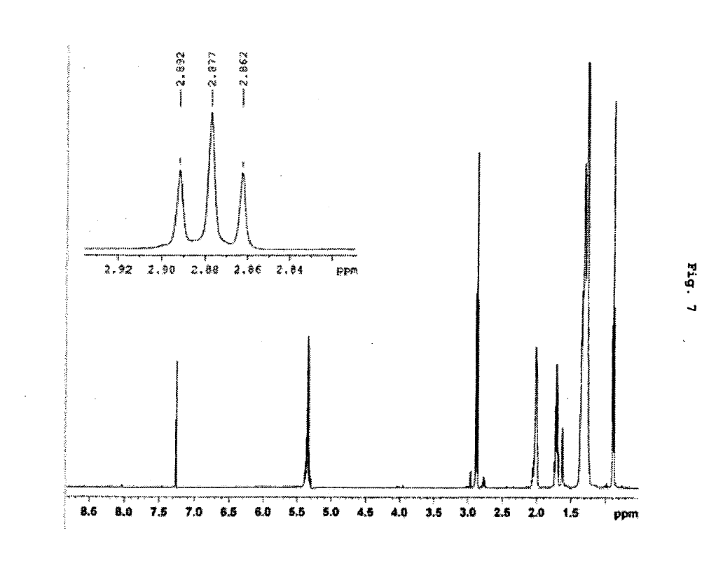

FIG. 7: the proton NMR spectrum at 500 MHz (CDCl₃) of olive FA chlorides,

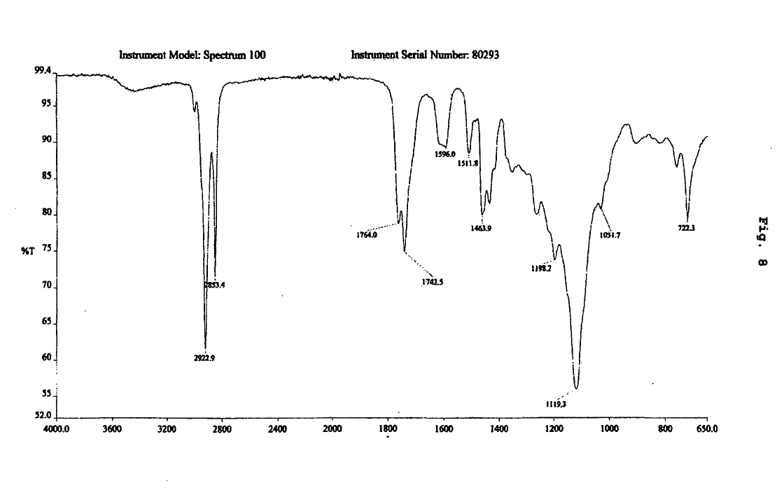

FIG. 8: the FT-IR spectrum of flavanolic polyphenols from grapeseed, alkylated and stabilized with olive oil FAs,

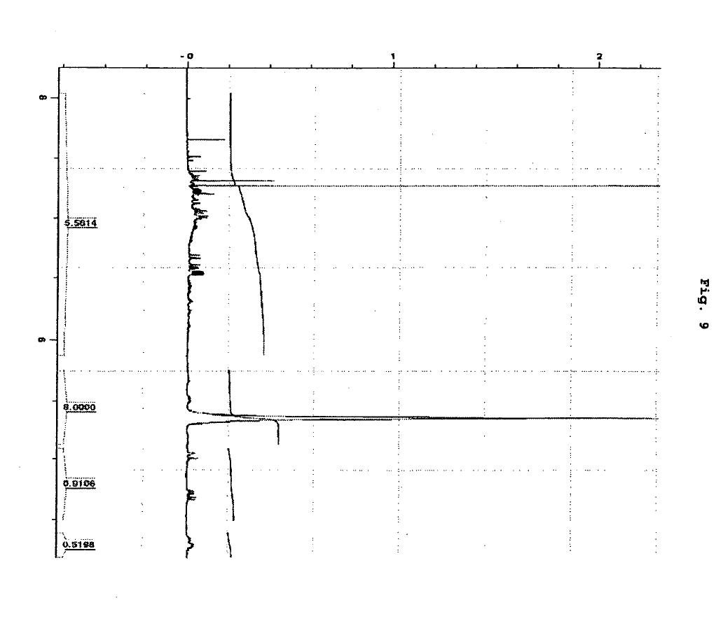

FIG. 9: the downfield portion of the ¹H NMR spectrum (500 MHz, CDCl₃) of alkylated grape seed flavanolic polyphenols stabilized with olive oil FAs, and integration curves,

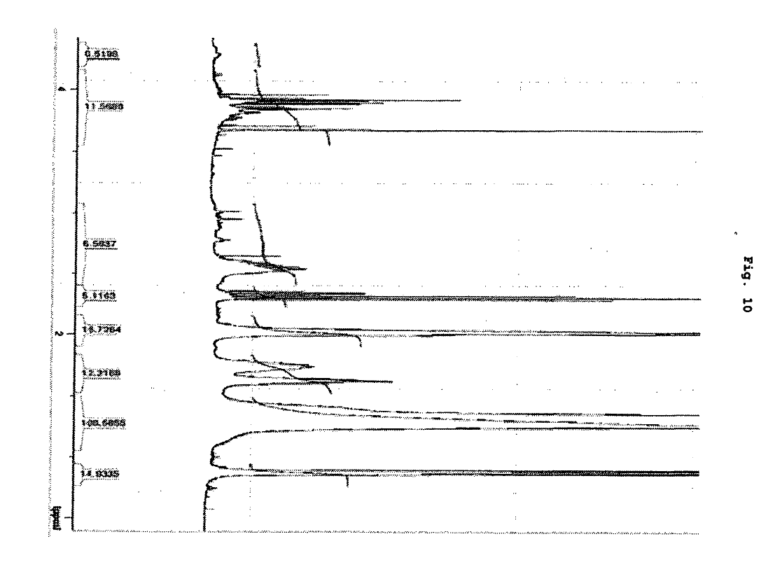

FIG. 10: the upfield portion of the ¹H NMR spectrum (500 MHz, CDCl₃) of alkylated grape seed flavanolic polyphenols stabilized with olive oil FAs, and integration curves,

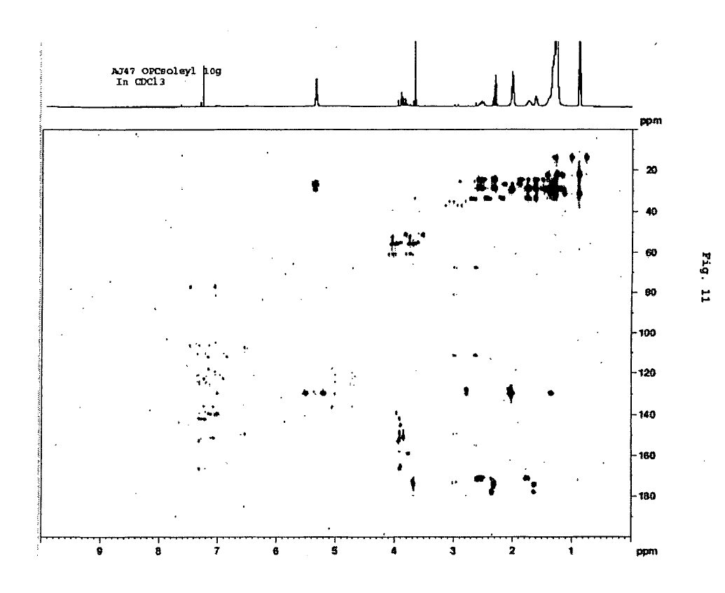

FIG. 11: the ¹H-¹³C HMBC 2D NMR spectrum (500 MHz, CDCl₃) of flavanolic polyphenols from grapeseed, alkylated and stabilized with olive oil FAs.

### EXAMPLE 1

### Step of O-Alkylation of Catechin

50 mg (0.172 mmol) of catechin are dissolved in 5 ml of anhydrous acetone in a double-necked flask with a top-mounted condenser. With stirring under an argon atmosphere, in the presence of 23.8 mg (0.172 mmol, 2 chemical eq) of potassium carbonate (K₂CO₃), 8.3 μL (0.086 mmol=2 chemical eq) of dimethyl sulfate (DMS) are added. The reaction is heated at reflux for 27 hours.

The reaction mixture is filtered on a No. 4 frit to remove the K₂CO₃, and the acetone is evaporated. The residue is taken up in 20 ml of ethyl acetate. The organic phase is washed with 2 times 20 ml of water, dried over sodium sulfate, filtered and evaporated to dryness to leave a residue of 48 mg (crude yield=91.6%, on the basis of monomethylated derivatives, mw=304).

The mixture obtained is analyzed by High Performance Liquid Chromatography on a reversed-phase column (C18), with detection by mass spectrometry at atmospheric pressure and electrospray ionization (HPLC-ESI-MS), shown in FIG. 1. In evidence are the most intense ion currents, of mass characteristic of monomethylated catechins ([M+H]⁺=305), at retention times (TR)=15.79; 15.95; 17.75; and 17.84 minutes, and minority ion currents, of mass ([M+H]⁺=319), at TR=21.66; 23.66; 24.67; 26.02; and 27.34 minutes, corresponding to the various dimethylated catechins.

### EXAMPLE 2

### Step of O-Alkylation of Flavonoid Polyphenol

31.18 g (“108 mmol”, expressed in “catechol” units) of grape seed polyphenol extract are placed in solution in 120 ml of aprotic solvent (anhydrous acetone), in the presence of 6 chemical equivalents of potassium carbonate (44.64 g=646 mmol). The resulting suspension is heated at reflux, with stirring and under an argon atmosphere, in a one-liter three-neck round-bottom flask fitted with a condenser.

Using a dropping funnel, 7.65 ml of the methyl donor (dimethyl sulfate, 81.5 mmol; each mole of DMS liberating 2 mol of “methyl”=2×81.5=163 equivalents, or =1.5 chemical equivalent/mass of polyphenol extract employed), or isopropyl donor (2-iodopropane), are added dropwise over a time of 15 minutes.

The chemical equivalents are calculated by counting a “maximum” average of 4 alkylatable phenolic hydroxyl residues per “flavanol unit”. Hence it is considered that each portion of 290 g of extract corresponds to 1 mol of catechin, which possesses four phenol functions of which only one, or even two, is (are) to be converted to methyl or isopropyl ether(s). The chemical equivalent of the alkylating reagent is therefore equal to a quarter of the number of moles of “catechin” present in the extract employed.

After eight hours at reflux under an argon atmosphere, the reaction is cooled. Following addition of a tenth-concentration hydrochloric acid solution, until an acid pH is obtained (540 ml), stirring is continued for 45 minutes more. The reaction mixture is concentrated under vacuum (evaporation of the acetone). The residual aqueous phase is extracted with an equal volume of ethyl acetate, which is washed with two times 400 ml of distilled water (until the washing water is neutral). This organic phase is then dried over anhydrous sodium sulfate, filtered, and evaporated under reduced pressure, to leave the residue of the alkylated polyphenols (20.88 g; crude yield=63.9%).

In the preferred case where each molecule of the initial extract undergoes a single methylation per flavanolic unit (“catechin”), a mixture of the various possible regioisomers and stereoisomers is obtained, such as the monomers and dimers featured below in the formulae IX to XXVI.

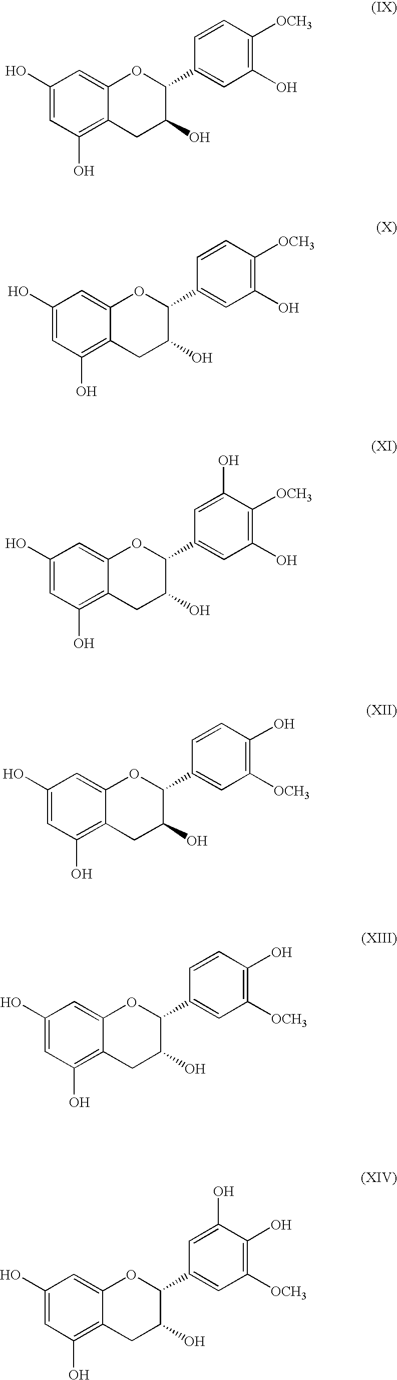

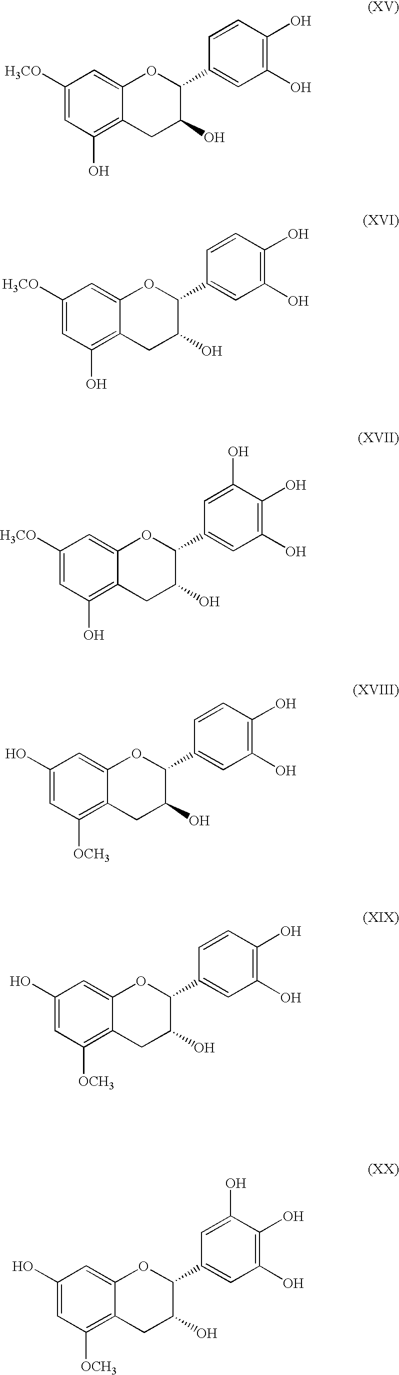

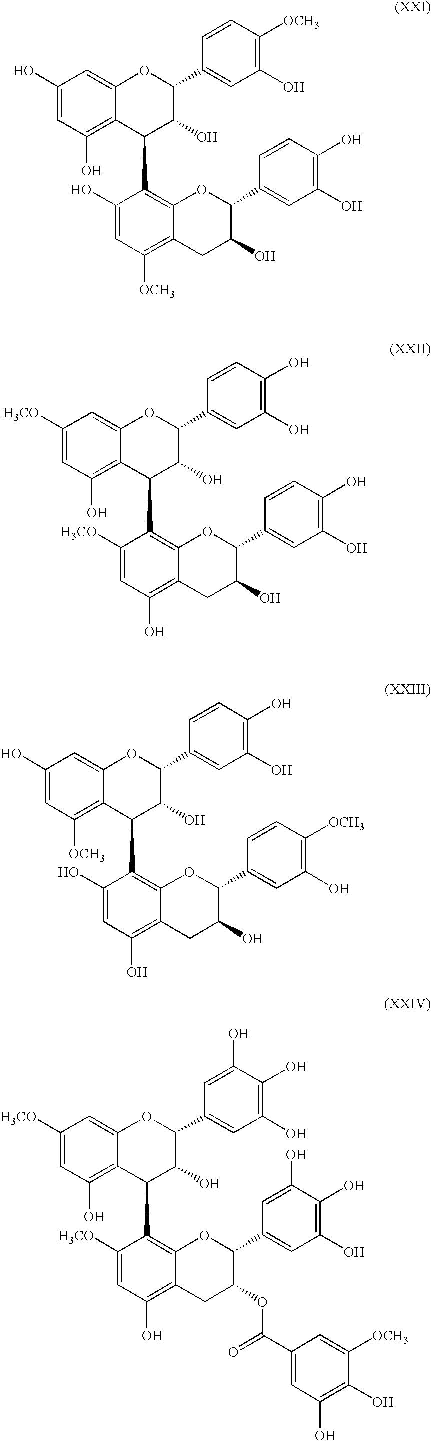

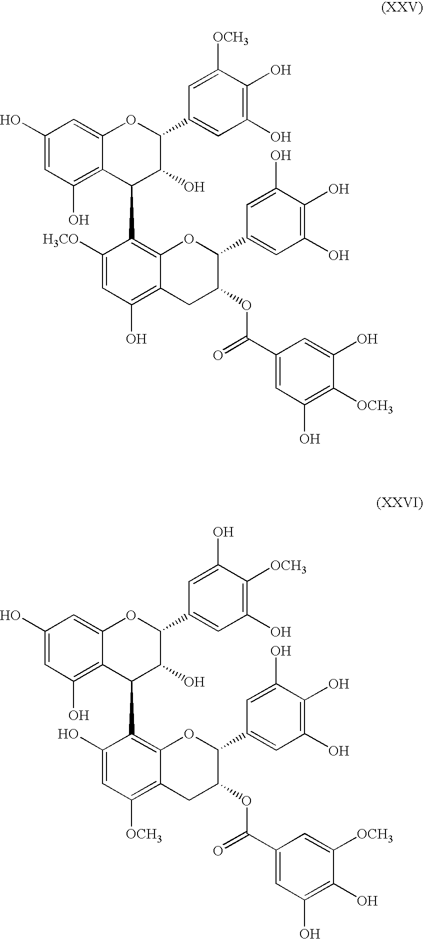

As for the preceding example, the alkylated (methylated) structures of these flavonoid compounds are deduced from the analysis of their various spectra:
The presence of phenolic methyl ethers is manifested in IR (FIG. 2), in particular, by the appearance of absorption bands between 2974 and 2836 cm⁻¹ which are characteristic of methyl C—H (elongation) and, between 1064 and 1035 cm⁻¹, those which are characteristic of ether (C—O) functions.

The HMBC 2D NMR spectrum shows correlations between oxygen-bearing aromatic carbons (from 148 to 160 ppm) and the protons of methyl ethers, which resonate from 3.7 to 3.94 ppm. An expansion of this zone is inserted into the overall spectrum shown in FIG. 3.

### EXAMPLE 3

### Preparation of Acylating Agents

### Step 1: Saponification of Olive Oil:

50.46 g of “virgin” olive oil (57 mmol, =“171 eq”), placed in a round-bottom flask equipped with a condenser, are admixed with 16.08 g of potassium hydroxide (285 mmol, 1.67 eq) in solution in 2.5 ml of ethanol and 50 ml of water. The reaction is taken to reflux for 5 hours. It is stirred for a further 14 h, at ambient temperature.

After the resulting solution has been extended with 300 ml of water, tenth-concentration (3.7%; w/v) hydrochloric acid is added until an acid pH is obtained in the aqueous phase (approximately 250 ml). The contents of the round-bottom flask, which comprises a pasty “insoluble” product at the surface, are then transferred to a separating flask and extracted with 700 ml of hexane. The organic phase is separated off and then washed with 2 times 300 ml of distilled water (to give a neutral pH of this aqueous phase).

The organic phase is dried over sodium sulfate, filtered on a No. 4 frit, and then evaporated to yield a residue of 42.9 g (crude yield=88.8%).

The infrared spectrum recorded in ATR mode with Fourier transform (FIG. 4) shows a band which is characteristic of free organic acids at 1709 cm⁻¹, along with the disappearance of the ester bands of the starting oil.

Step 2: Activation of Fatty Acids Obtained from the Saponification of Olive Oil by Formation of Chlorides:

The solution of 41.5 g of free fatty acids (147.1 mmol) obtained from step 1 in 232 ml of chloroform (stabilized on amylene) is stirred under an argon atmosphere in a round-bottom flask which is cooled by an ice bath. Using a dropping funnel, 13.8 ml of oxalyl chloride (162 mM=1.1 eq) are introduced dropwise over a period of 30 minutes. 1 ml of dimethylformamide (DMF) is introduced, and stirring is continued over the ice bath for 5 minutes. Concentration of the reaction mixture under reduced pressure (chloroform and oxalyl chloride in excess) then gives 44.3 g of an oily residue with a slight yellow coloration (crude yield=100%).

By distillation in a ball oven (kugelrohr) under a high vacuum (2 mmHg), this residue is decolorized (colorless liquid), by collecting the fractions which distill at from 178 to 195° C.

In order to analyze the composition of the mixture of fatty acid chlorides obtained, a few microliters of distillate are exposed to methanol. The total mixture is then injected into a gas chromatograph equipped with a “FAME” (fatty acid methyl ester) column and an online mass detector (DSQ-II). In the chromatogram shown in FIG. 5, the peak at 17.8 min corresponds to the stearate (M+•=298), that at 18.07 min to the oleate (M+•=296), that at 18.08 min to a linoleate (M+•=294), and that at 19.38 min to the linolenate (M+•=292). Their relative intensities are a good indication of their respective proportions.

The FT-IR (FIG. 6) and proton NMR (FIG. 7) spectra are in perfect agreement with the exclusive formation of these chlorides:
A band at 1798 cm⁻¹, characteristic of acyl chlorides.
The protons alpha to the carbonyl (t, J=7.5 Hz) exhibit a chemical shift at 2.9 ppm, which is characteristic of the conversion of carboxyls to acid chlorides.

### EXAMPLE 4

### Acylation of the Alkylated Flavonoid Extract of Grape Seeds

21.93 g (72 mmol=288 chemical eq.) of alkylated (methylated) grape seed flavonoid extract, according to example 2, are partially dissolved in 270 ml of chloro-form (stabilized with amylene) and are placed under an argon atmosphere. They are admixed with the basic agent, triethylamine (40.56 ml=29.45 g (d=0.726)=291.5 mmol=1 chemical eq.), and the “solution” is treated with ultrasound for 5 minutes. With magnetic stirring and at ambient temperature, using a dropping funnel, 87.55 g of acylating agents as prepared in example 3 (olive oil FA chlorides=288 mmol=1 chemical eq.) in dilution in 60 ml of chloroform are added dropwise over a period of 20 minutes. Each falling drop produces an evolution of gas.

The reaction is left for a further seven hours with stirring at ambient temperature, before being placed in a separating funnel and washed with 190 ml of tenth-concentration hydrochloric acid, 90 ml of a 10% (w/v) NaHCO₃ solution in water, and finally with distilled water until neutrality (three times 90 ml). The organic phase is dried over sodium sulfate, filtered, then evaporated to dryness, under reduced pressure. It leaves a residue of 67.27 g of stabilized and alkylated grapeseed active flavonoid substances (=49.68 mmol; crude yield=69%, average mw=1354).

With the aim of obtaining means of identifying these active substances, the whole product is then subjected to the maximum spectral measurements:
The Fourier-transform infrared spectrum acquired in ATR mode ( ) shows the appearance of an intense band at 1764 cm⁻¹, which is characteristic of the carboxyls of phenolic esters, simultaneous with the disappearance of the broad band centered on 3350 cm⁻¹, which corresponded to the free phenolic hydroxyls.
The proton NMR spectrum (500 MHz, CDCl₃) is shown with its integral curves, in 2 parts. Downfield (FIG. 9), it allows “numbering” of the proportion of the aromatic protons=5.5 (region from 7.95 to 5.90 ppm), relative to the olefinic protons: complex centered on 5.35 ppm, calibrated at 8 protons (in agreement with an average of four olefins per catechol unit). Upfield (FIG. 10), it allows observation of the singlet signals of the methoxys of aromatic ethers (from 4.05 to 3.58 ppm) and the complex of signals which are characteristic of methylenic protons alpha to the carboxyls of aromatic esters, centered on δ=2.49 ppm.

The long-distance ¹H-¹³C heteronuclear two-dimensional NMR spectrum at 500 MHz (FIG. 11), obtained in inverse mode (HMBC), clearly shows the correlations which are in perfect agreement with the diversified structures of flavanolic polyphenols which are alkylated (methyl ethers of aromatic oxygens) and esterified (predominantly unsaturated fatty acid esters, in statistical mixture as resulting from the olive oil used for preparing the acylating agents phenols and alicyclic alcohols).

In the preferred case in which each molecule of the initial extract has undergone only one methylation per flavanolic unit (“catechin”), and in which the residual phenolic functions and the flavanolic alcohol are all acylated by the olive oil FA mixture, a mixture is obtained of the various possible regioisomers and stereoisomers of monomers and dimers that are featured below in the formulae XXVII to XXXI:

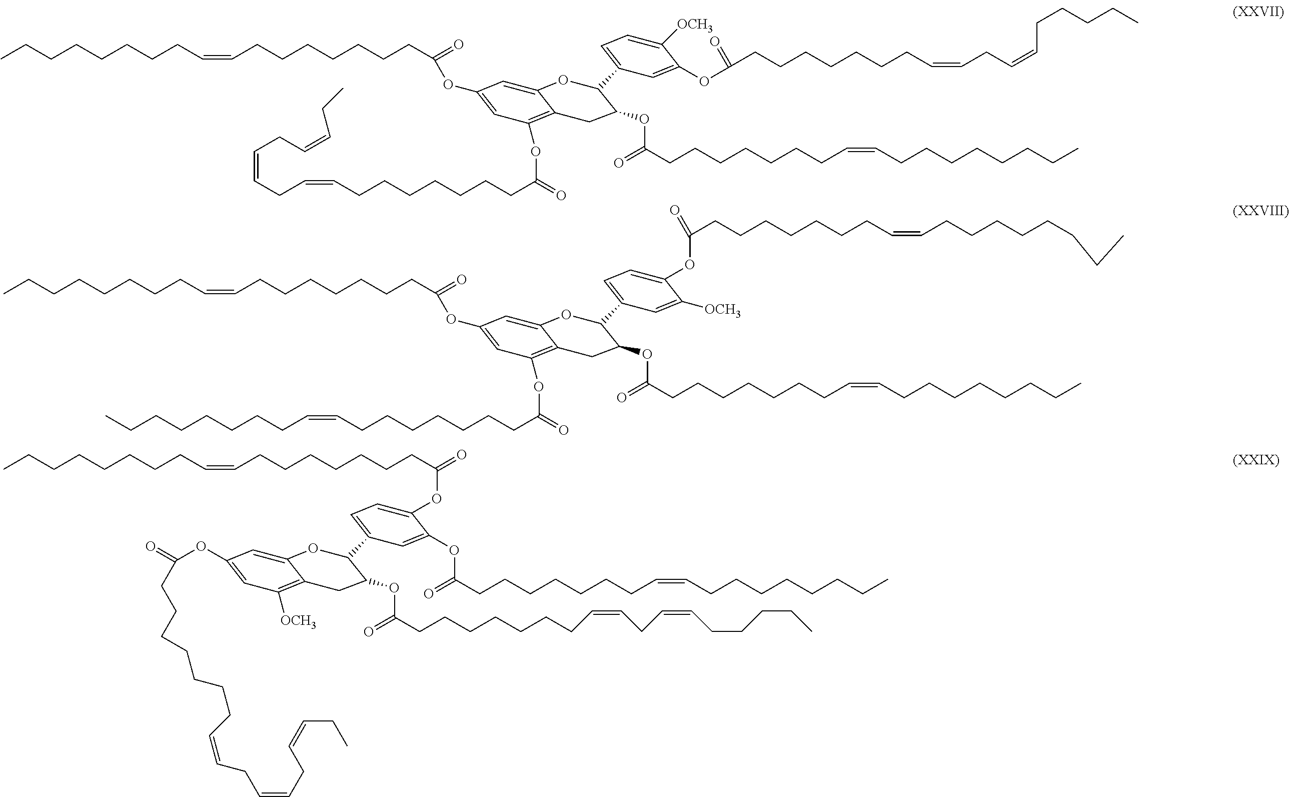

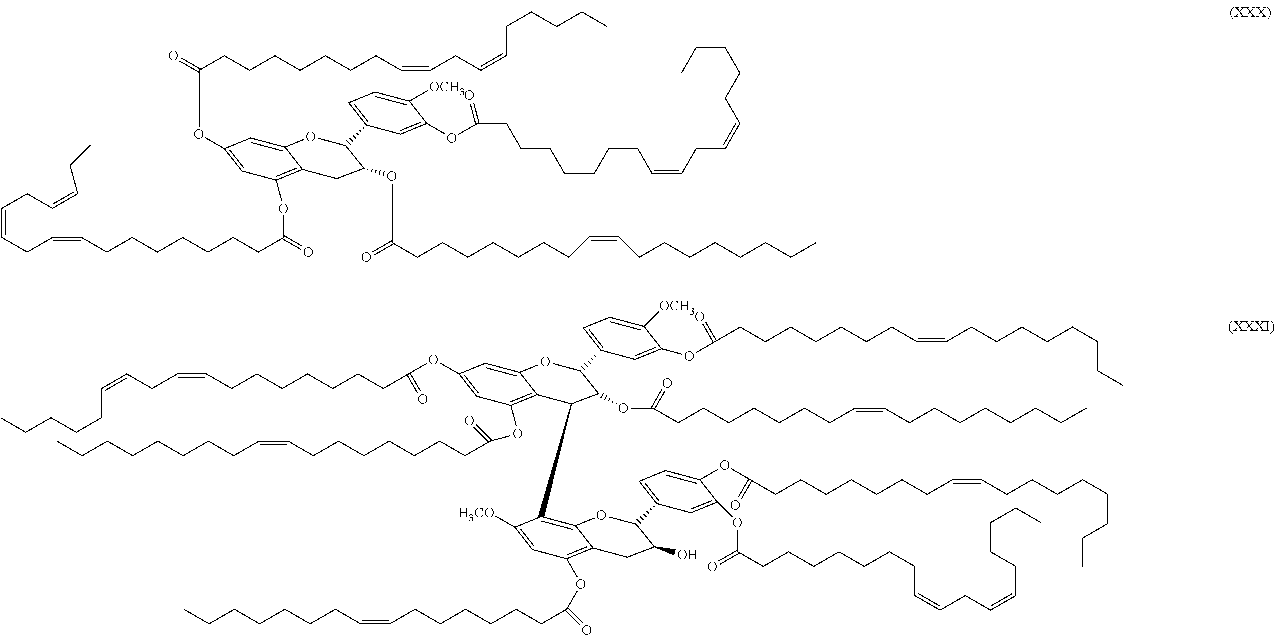

### EXAMPLE 5

### Cosmetic Formulations

FORMULA A

| PHASES | STARTING MATERIALS | % |
|---|---|---|
| 101 | Water | 80.8000 |
| 102 | Tetrasodium EDTA | 0.0500 |
| 103 | Glycerol | 5.0000 |
| 104 | Carbomer | 0.3500 |
| 201 | Wheat cetearyl glycosides | 0.7500 |
| 202 | Barley cetearyl glycosides | 1.7500 |
| 203 | Cetearyl alcohol | 2.5000 |
| 204 | Composition of the invention | 0.05 to 1 |
| 205 | Butyrospermum parkii | 2.5000 |
| 206 | Tocopheryl acetate | 0.5000 |
| 207 | Grapeseed oil (Vitis vinifera) | 3.0000 |
| 208 | Cetyl alcohol | 1.0000 |
| 209 | Potassium cetyl phosphate | 1.0000 |
| 301 | Preservatives | 0.6000 |
| 401 | Fragrance | 0.2000 |
| 501 | Sodium hydroxide qs pH 6.00 | |

FORMULA B

| PHASES | STARTING MATERIALS | % |
|---|---|---|
| 101 | Water | 79.40000 |
| 102 | Tetrasodium EDTA | 0.05000 |
| 103 | Citric acid qs final pH 5.5 | 0.15000 |
| 201 | Xanthan gum | 0.30000 |
| 202 | Butylene glycol | 5.00000 |
| 301 | Ceteareth-20 | 1.50000 |
| 302 | Glyceryl stearate | 2.00000 |
| 303 | Composition of the invention | 0.05 to 1 |
| 304 | Butyrospermum parkii butter | 1.00000 |
| 305 | Hexyl laurate | 4.00000 |
| 306 | Dimethicone | 3.00000 |
| 307 | Squalane | 2.00000 |
| 308 | Tocopheryl acetate | 0.50000 |
| 401 | Preservatives | 0.60000 |
| 501 | Fragrance | 0.50000 |

1. Compositions of polyphenol derivatives, characterized in that said polyphenol derivatives comprise monomers, oligomers or polymers of units conforming to the formula (I): 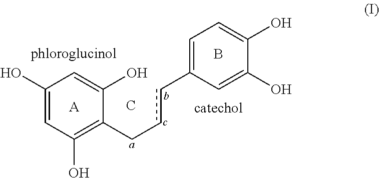
these units being characterized by the simultaneous presence of a phloroglucinol nucleus (nucleus A) and of a catechol nucleus (nucleus B), which are joined to one another by a 3-carbon segment such as C, said derivatives being overactivated, with regard to their nucleophilic power, by alkylation of at least one phenolic function of each unit, and being stabilized by esterification with mixtures of predominantly unsaturated fatty acids (UFA) of all of the other hydroxyl (phenolic and alcoholic) functions.

2. The compositions according to claim 1, characterized in that in said units the nucleus A of the polyphenols is fused to a further oxygen-containing heterocycle by formation of a bond of one of its oxygens with the carbon b of the segment C, as in the case of the flavonoid backbone of formula (II)

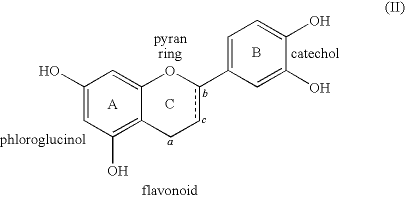

3. The compositions according to claim 1, characterized in that in said units the 3 carbons of the segment C of said polyphenols are sp2 hybridized (double bond between b and c and carbonyl at a) as for quercetin of formula (III) 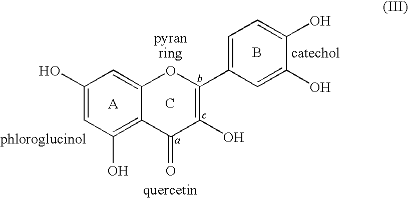
or a double bond is formed between a and c and carbonyl at b, as for cyanidol of formula (IV) 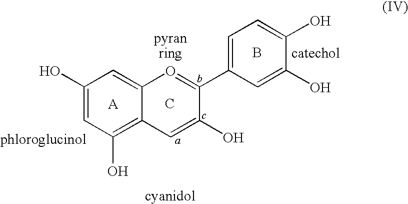
or the carbon a is sp3 hybridized alone, or all 3 are sp3 hybridized, as in the case of catechin of formula (V) 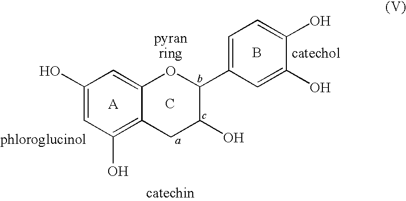
it then being possible for the carbon a of the segment C to act as a point of attachment with the A rings of the other units to form the oligomers or the polymers.

4. The compositions according to claim 1, characterized in that the number of —O-alkyl groups per unit is not equal to the number of hydroxyls present on average per unit.

5. The compositions according to claim 4, characterized in that the number of hydroxyls present on average per unit is 1 or 2.

6. The compositions according to claim 1, characterized in that the alkyl group or groups are methyl, isopropyl or tert-butyl groups.

7. The compositions according to claim 1, characterized in that said esters are fatty acid esters of vegetable oils.

8. The compositions according to claim 7, characterized in that these esters comprise the radicals R corresponding to saturated fatty acids, such as stearic acid, to monounsaturated fatty acids, such as oleic acid, and to essential polyunsaturated fatty acids, such as linoleic and linolenic acids.

9. The compositions according to claim 7, characterized in that the vegetable oils are selected from olive oil or grapeseed oil.

10. The compositions according to claim 1, characterized in that said unitary derivatives conform to the formula (VI): 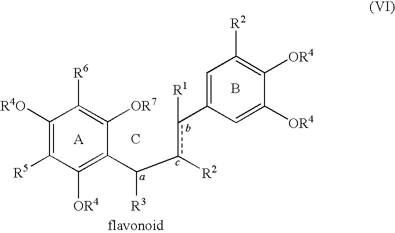
in which
R¹ is a hydrogen or the junction point at R⁷ of a single unit,
R² is a hydrogen, or an O-acyl radical of an unsaturated fatty acid from a vegetable oil,
R³ is a hydrogen, a carbonyl or the junction point at R⁵ or at R⁶ of another unit,
R⁴ is an alkyl radical, or an acyl radical of an unsaturated fatty acid of a vegetable oil, represented by R as defined above,
R⁵ is a hydrogen or the junction point at R³ of another unit, directly or through a carbon entity (methylene, methylmethine, etc.),
R⁶ is a hydrogen or the junction point at R³ of another unit, directly or through a carbon entity (methylene, methylmethine, etc.),
R⁷ is an alkyl radical or an acyl radical of a fatty acid of a vegetable oil, represented by R, or the junction point at R¹ of the same unit,
and the diastereoisomers and regioisomers of these moieties.

11. The compositions according to claim 10, characterized in that said derivatives are derivatives of the dimer of catechin (B3) and of the trimer of epicatechin (C2), of formulae (VII) and (VIII):

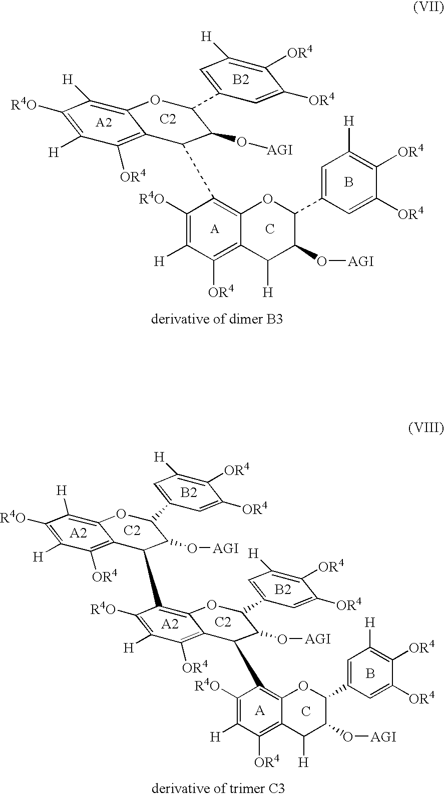

12. The compositions according to claim 1, characterized in that said derivatives correspond to stabilized and alkylated derivatives of plant extracts.

13. The compositions according to claim 12, characterized in that said plant extracts are vine extracts fermented or green tea, fresh or roasted cocoa beans, or of pine.

14. The compositions according to claim 13, characterized in that said vine extracts are obtained from grape seeds or grape marcs.

15. A process for preparing compositions according to claim 1, in that it comprises the reaction of the polyphenol compositions formed of units,
in a first step, with an alkylating agent under conditions allowing substitution of an alkyl group for the hydrogen of at least 1 phenolic OH group per constituent monomeric unit of each molecule, preferably of 1 to 2, and
in a second step, with an acylating agent, especially an acid anhydride or acid chloride, under conditions allowing substitution by a mixture of acyl radicals —COR liberated by the acylating agent, R for the hydrogen of the —OH groups which are still free after alkylation.

16. The process according to claim 15, characterized in that the acylating agent is obtained from a vegetable oil by a process comprising:
the saponification of the glycerides of the vegetable oil, followed by an acidification,
activation by dehydration where the acylating agent is an acid anhydride, or by chloridation where it is an acid chloride.

17. Cosmetic compositions characterized in that they comprise an amount effective for combating skin aging of one or more compositions of polyphenol derivatives according to claim 1, in combination with inert vehicles appropriate for external use.

18. The compositions according to claim 17, characterized in that they take a form appropriate for topical administration, such as cream, ointment, emulsion, gel, liposomes, lotion.

19. The compositions according to claim 17, characterized in that they contain from 0.5% to 5% of active product, preferably from 2% to 3%.

20. The application of the compositions according to claim 1 in dietetics.

21. The application according to claim 20, characterized in that said compositions are added to drinks, as for example to fruit juices, tonic drinks, to dairy products and derivatives such as butter, in liquid form, or else as granules or the like, gels, or in paste form, incorporated, for example, into confectionery such as fruit gums, candy, chewing gums.

22. The compositions according to claim 1 for use as medicaments.

23. Pharmaceutical compositions characterized in that they comprise a therapeutically effective amount of at least one composition according to claim 1, in combination with a pharmaceutically acceptable vehicle.

24. The compositions according to claim 22, characterized in that they take a form appropriate for administration by oral, topical or parenteral administration.

25. The compositions according to claim 24, characterized in that they take a form for oral administration, such as solution, tablet, gel capsule or syrup.

26. The compositions according to claim 24, characterized in that it takes a form for topical administration, such as cream, ointment, gels, lotions or patch.

27. The compositions according to claim 24, characterized in that it takes a form for parenteral administration, such as a sterile or sterilizable injectable solution.
